(19) United States
(12) Patent Application Publication
(10) Pub. No.: US 2021/0017223 A1
(43) Pub. Date: Jan. 21, 2021

(54) Separation Method

(71) Applicant: Cytiva BioProcess R&D AB, Uppsala (SE)

(72) Inventors: Tomas Bjorkman, Uppsala (SE); Bengt Westerlund, Uppsala (SE)

(21) Appl. No.: 16/981,716

(22) PCT Filed: Mar. 26, 2019
(86) PCT No.: PCT/EP2019/057587

§ 371 Date: Sep. 17, 2020

(30) Foreign Application Priority Data
Mar. 29, 2018 (GB) 1805142.5

(51) Int. Cl. C07K 1/36; C07K 1/22; C07K 1/18; C07K 16/00

(52) U.S. Cl. CPC C07K 1/36; C07K 16/00; C07K 1/18; C07K 1/22

(57) ABSTRACT

The invention relates to a method of separating immunoglobulin variants, comprising the steps of: a) providing a column packed with an Fc-binding affinity chromatography resin; b) loading a sample comprising at least two Fc-comprising immunoglobulin variants onto the column; c) optionally washing the column with a washing liquid; and d) conveying an eluent through said column to elute at least a target immunoglobulin variant from said column and recovering one or more eluate fractions comprising the target immunoglobulin variant in enriched form.

### TECHNICAL FIELD OF THE INVENTION

The present invention relates to the field of affinity chromatography, and more specifically to differential separations of immunoglobulins with different affinity to Protein A, using affinity separation resins with mutated domains of Protein A.

### BACKGROUND OF THE INVENTION

Immunoglobulins represent the most prevalent biopharmaceutical products in either manufacture or development worldwide. The high commercial demand for and hence value of this particular therapeutic market has led to the emphasis being placed on pharmaceutical companies to maximize the productivity of their respective mAb manufacturing processes whilst controlling the associated costs.

Affinity chromatography is used in most cases, as one of the key steps in the purification of these immunoglobulin molecules, such as monoclonal or polyclonal antibodies. A particularly interesting class of affinity reagents is proteins capable of specific binding to invariable parts of an immunoglobulin molecule, such interaction being independent on the antigen-binding specificity of the antibody. Such reagents can be widely used for affinity chromatography recovery of immunoglobulins from different samples such as but not limited to serum or plasma preparations or cell culture derived feedstocks. An example of such a protein is staphylococcal protein A, containing domains capable of binding to the Fc and Fab portions of IgG immunoglobulins from different species. These domains are commonly denoted as the E-, D-, A-, B- and C-domains.

Staphylococcal protein A (SpA) based reagents have due to their high affinity and selectivity found a widespread use in the field of biotechnology, e.g. in affinity chromatography for capture and purification of antibodies as well as for detection or quantification. At present, SpA-based affinity medium probably is the most widely used affinity medium for isolation of monoclonal antibodies and their fragments from different samples including industrial cell culture supernatants. Accordingly, various matrices comprising protein A-ligands are commercially available, for example, in the form of recombinant protein A (e.g. MabSelect™, GE Healthcare) and of mutated alkali-stable protein A variants (e.g. MabSelect SuRe and MabSelect PrismA, GE Healthcare).

These protein A matrices are normally used for separation of immunoglobulins from other proteins present in cell cultures. There is however also a need for separation between different variants of immunoglobulins present in a cell broth. This applies in particular to bi- and multispecific antibodies which are expressed together with significant amounts of non-desired immunoglobulin variants, such as half-antibodies, homodimeric antibodies etc.

### SUMMARY OF THE INVENTION

One aspect of the invention is to provide a method of separating immunoglobulin variants directly in the affinity capture step. This is achieved by a method comprising the steps of:

a) providing a column packed with an Fc-binding affinity chromatography resin;
b) loading a sample comprising at least two Fc-comprising immunoglobulin variants onto the column;
c) optionally washing the column with a washing liquid; and
d) conveying an eluent through the column to elute at least a target immunoglobulin variant from the column and recovering one or more eluate fractions comprising the target immunoglobulin variant, suitably in enriched form.

Preferably, the steps are performed in the order as listed above.

### Definitions

The terms “antibody” and “immunoglobulin” are used interchangeably herein, and are understood to include also fragments of antibodies, fusion proteins comprising antibodies or antibody fragments and conjugates comprising antibodies or antibody fragments. See below for a detailed discussion of example antibodies and immunoglobulins encompassed by the invention.

The terms an “Fc-binding polypeptide” and “Fc-binding protein” mean a polypeptide or protein respectively, capable of binding to the crystallisable part (Fc) of an antibody and includes e.g.

Protein A and Protein G, or any fragment or fusion protein thereof that has maintained said binding property.

As used herein, the terms “comprises,” “comprising,” “containing,” “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

In the absence of specific temperature data indicating otherwise, all measurements and methods are performed at room temperature (22+/−2° C.).

### BRIEF DESCRIPTION OF FIGURES

FIG. 1 shows a) an exemplary intact antibody/immunoglobulin and b) an exemplary half-antibody.

FIG. 2 shows a SEC chromatogram of the antibody feed used in the separation examples. The full antibody (A) eluted at 14.30-14.55 ml and the half-antibody (B) at 15.5 ml. The chromatogram also shows the presence of significant amounts of lower Mw material (host cell proteins etc.) in the feed.

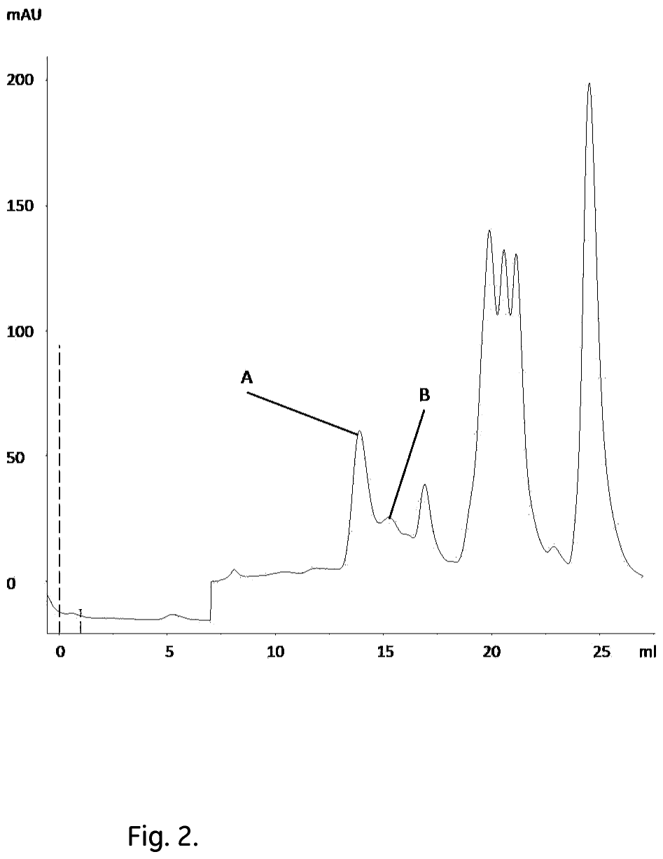

FIG. 3 shows a) the elution peak (pH 6.0-3.0 gradient) of the feed containing both an intact antibody and a half antibody on the protein A resin MabSelect™ SuRe™ LX and b) the composition with respect to intact antibody, half antibody and aggregates of fractions from the peak in a).

FIG. 4 shows a) the elution peak (pH 6.0-3.0 gradient) of the feed containing both an intact antibody and a half antibody on the protein A resin MabSelect SuRe pcc and b) the composition with respect to intact antibody, half antibody and aggregates of fractions from the peak in a).

FIG. 5 shows a) the elution peak (pH 6.0-3.0 gradient) of the feed containing both an intact antibody and a half antibody on the protein A resin MabSelect SuRe PrismA™ and b) the composition with respect to intact antibody, half antibody and aggregates of fractions from the peak in a).

FIG. 6 shows a) the elution peak (pH 5.0-3.0 gradient) of the feed containing both an intact antibody and a half antibody on the protein A resin MabSelect SuRe PrismA and b) the composition with respect to intact antibody, half antibody and aggregates of fractions from the peak in a).

FIG. 7 shows a) the elution peak (pH 4.5-3.0 gradient) of the feed containing both an intact antibody and a half antibody on the protein A resin MabSelect SuRe PrismA and b) the composition with respect to intact antibody, half antibody and aggregates of fractions from the peak in a).

FIG. 8 shows a) a multimodal cation exchange (Capto™ MMC ImpRes™) chromatogram of the whole antibody fraction of the Example 2 eluate and b) a SEC chromatogram of the two peaks in the chromatogram.

FIG. 9 shows a) a multimodal cation exchange (Capto™ MMC ImpRes) chromatogram of a mixture of the whole and half antibody fractions of the Example 2 eluate and b) SEC analysis of the fractions in the chromatogram.

FIG. 10 shows a) a cation exchange (Capto™ SP ImpRes) chromatogram of a mixture of the whole and half antibody fractions of the Example 2 eluate and b) SEC analysis of the fractions in the chromatogram.

FIG. 11 shows a) an anion exchange (Capto™ Q) chromatogram of a mixture of the whole and half antibody fractions of the Example 2 eluate and b) SEC analysis of the fractions in the chromatogram.

FIG. 12 shows a) a HIC (Capto™ Butyl ImpRes) chromatogram (0.3M sodium sulphate-water gradient) of a mixture of the whole and half antibody fractions of the Example 2 eluate and b) SEC analysis of the fractions in the chromatogram.

FIG. 13 shows a) a HIC (Capto™ Butyl ImpRes) chromatogram (0.3M ammonium sulphate-water gradient) of a mixture of the whole and half antibody fractions of the Example 2 eluate and b) SEC analysis of the fractions in the chromatogram.

### DETAILED DESCRIPTION

The invention discloses a method of separating immunoglobulin variants. This method comprises the steps of:

a) Providing a column packed with an Fc-binding affinity chromatography resin. The Fc-binding affinity chromatography resin can suitably be a Protein A resin, as exemplified by a number of commercially available products, e.g. MabSelect™ and MabSelect Xtra (GE Healthcare), ProSep™ (Merck-Millipore), Praesto™ AP (Purolite), Toyopearl™ AF (Tosoh) etc. The Protein A resin can suitably be an alkali-stable resin comprising mutated Protein A variants, as exemplified by MabSelect SuRe, MabSelect SuRe LX, MabSelect SuRe pcc and MabSelect PrismA (all GE Healthcare). Particularly suitable are particulate Fc-binding (e.g. Protein A) resins with a volume-weighted median particle diameter (d50,v) of less than 70 micrometers, such as 40-65 or 45-65 micrometers. Examples of such resins are MabSelect SuRe pcc (d50,v at 50 μm) and MabSelect PrismA (d50,v at 60 μm). d50,v can conveniently be measured by light diffraction, such as in a Mastersizer instrument (Malvern). The low particle size improves the resolution of the separation, while still allowing process-scale chromatography. Alternatively, the Fc-binding affinity resin can be one or more membranes with Fc-binding affinity ligands (e.g. Protein A) coupled to the membrane pore surfaces. Examples of such membranes are given in WO 2018/011600 A1, which is hereby incorporated by reference in its entirety.
b) Loading a sample comprising at least two Fc-comprising immunoglobulin variants onto the column. One of the Fc-comprising immunoglobulin variants can e.g. be an intact immunoglobulin, while another one of the Fc-comprising immunoglobulin variants can e.g. be an Fc-comprising fragment of an immunoglobulin. An example of an intact immunoglobulin can be a full antibody with an Fc region and at least two Fab regions, while an example of a fragment can be a half-antibody with an Fc region and only one Fab region. The sample can suitably be a cell culture supernatant, which in addition to the immunoglobulin variants may also comprise e.g. host cell proteins, DNA, culture medium components and aggregated immunoglobulins. pH and conductivity may be close to physiological conditions, e.g. pH 6.5-8 and 5-20 mS/cm. The sample can e.g. be a clarified supernatant harvested from a cell culture (e.g. CHO cells or other immunoglobulin-expressing cells), or it can be a filtrate obtained from a continuous or semicontinuous perfusion culture. To increase the throughput, the total amount of Fc-comprising immunoglobulin variants loaded onto the column may exceed the dynamic immunoglobulin binding capacity (at 10% breakthrough) of the column, typically measured at the same residence time as used in the loading procedure, e.g. about 4 or 6 min. This is particularly suitable where a substantial portion of the Fc-comprising immunoglobulin variants in the feed are undesired variants (e.g. half-antibodies) with a lower avidity/affinity to the column than the desired variant (e.g. a full antibody). The undesired variants will then initially bind to the column but will be displaced by the more strongly binding desired variant, meaning that the column's binding capacity is not “wasted” on the undesired variants. Alternatively, the undesired variant may bind stronger than the desired variant, in which case the desired variant can be recovered from a flowthrough or a wash fraction. To provide for efficient displacement, the chromatography conditions may be manipulated to decrease the affinity somewhat. As an example, the pH during loading may be decreased to e.g. 4.5-5.5. The total amount of Fc-comprising immunoglobulin variants loaded onto the column can e.g. be at least 80, at least 90 or at least 100 mg per ml column bed volume, compared with the dynamic binding capacity of MabSelect PrismA, a high capacity resin, which is about 65 mg/ml IgG at 4 min residence time and about 80 mg/ml at 6 min residence time. The dynamic immunoglobulin binding capacity of a resin or column can be determined according to methods well known in the art. Typically, a 2 mg/ml solution of human polyclonal IgG (e.g. Gammanorm from Octapharma) in a pH 7.4 PBS buffer is pumped through the column at a rate producing the desired residence time and the protein concentration at the column outlet is monitored by measuring the 280 nm UV absorbance. When the outlet concentration reaches 10% of the loaded solution concentration, a 10% breakthrough is said to have occurred and the total amount of IgG loaded at this point is calculated and taken as the 10% breakthrough dynamic capacity.
c) Optionally washing the column with a washing liquid. The washing liquid can e.g. be a washing buffer as normally used in Protein A chromatography, i.e. a buffer with pH 5-8. The washing liquid may also comprise an additive for improving the washing efficiency, e.g. to improve the host cell protein clearance. Such additives are known in the art and may comprise one or more of a detergent, a water-miscible organic solvent, a chaotrope, arginine or an arginine derivative, calcium ions and tetraalkylammonium ions. The following documents describing suitable additives are hereby incorporated by reference in their entireties: U.S. Pat. Nos. 6,127,526, 6,870,034, 7,820,799, 7,834,162, 8,263,750, 7,714,111, 9,284,347, US20120283416, US20130197197, WO2014186350, WO2014192877, US20140094593, US20160108084 and US20160024147.
d) Conveying an eluent through the column to elute at least one of the immunoglobulin variants from the column. One or more eluate fractions can then be recovered, comprising a target immunoglobulin variant, suitably in enriched form, such as with a significantly higher ratio of target immunoglobulin variants to other immunoglobulin variants than in the feed. The eluent can suitably be a buffer with a pH low enough to allow elution of at least one of the immunoglobulin variants from the column, but not so low that extensive aggregation of the immunoglobulin(s) occurs. A typical elution pH can be within the 5.0-3.0 interval, such as 4.5-3.0, depending on the individual immunoglobulin variants. To obtain improved resolution between the variants, it can be advantageous to elute with a decreasing pH gradient, where a linear or non-linear gradient of decreasing pH is conveyed through the column. The start pH of the gradient can suitably be at least 4.5, such as at least 5.0 and the end pH can e.g. be 3.5 or lower, such as 3.0 or lower. Alternatively, the start pH can be at least 4.5 and the gradient is ended when the target immunoglobulin variant has eluted completely. In step d), an elution fraction comprising a target Fc-comprising immunoglobulin variant is suitably recovered and, in this fraction, the ratio of target Fc-comprising immunoglobulin variant concentration to total Fc-comprising immunoglobulin variant concentration is suitably higher than in the feed, such as at least 50% higher or at least 80% higher.

After step d), the method may comprise at least one further chromatography step e). Such a step can have the effect of decreasing concentrations of classical immunoglobulin contaminants such as host cell proteins and DNA, but it may also remove further undesired immunoglobulin variants. Several different chromatographic techniques can be used for this step. The step can e.g. be a cation exchange chromatography step, suitably in bind-elute mode with either pH (e.g. increasing pH) or conductivity (e.g. increasing conductivity) elution. The elution can suitably be with a continuous gradient or a step gradient. Commercially available cation exchange resins for use with such a step include e.g. Capto™ S, Capto SP ImpRes, SP Sepharose FF, SP Sepharose HP (all GE Healthcare), Eshmuno™ S, Fractogel™ SO3 (all Merck-Millipore), Toypearl™ SP (Tosoh) and Praesto™ SP (Purolite). Alternatively, the step can be a multimodal cation exchange step, suitably in bind-elute mode with e.g. pH elution (e.g. with an increasing pH gradient in the form of a continuous gradient or a step gradient). Commercially available multimodal cation exchange resins for use with such a step include e.g. Capto MMC, Capto MMC ImpRes (GE Healthcare) and Nuvia™ cPRIME (Bio-Rad). It is also possible to use an anion exchange chromatography step, e.g. in bind-elute mode with e.g. pH elution (e.g. with a decreasing pH gradient in the form of a continuous gradient or a step gradient). Commercially available anion exchange resins for use with such a step include e.g. Capto Q, Capto Q ImpRes, Q Sepharose FF, Q Sepharose HP (all GE Healthcare), Eshmuno Q, Fractogel TMAE (Merck-Millipore), Toyopearl Q (Tosoh) and Praesto Q (Purolite). Yet another possibility is to use a hydrophobic interaction chromatography (HIC) step, suitably in bind-elute mode with a decreasing salt gradient. Commercially available HIC resins for use with such a step include e.g. Capto Butyl, Capto Butyl ImpRes (GE Healthcare), Toyopearl Butyl (Tosoh). The further chromatography step e) may follow directly after step d), or after a step d′) of adjusting the buffer conditions to be compatible with the subsequent step. Such an adjustment can e.g. be an addition of salt to increase the conductivity and/or an addition of an acid or a base to decrease/increase pH. Alternatively, the adjustment can involve a buffer exchange, e.g. by diafiltration. Between steps d) and e) it is also possible to perform one or more other unit operations, such as virus inactivation, virus filtration, ultrafiltration/diafiltration etc.

### Immunoglobulin Variants

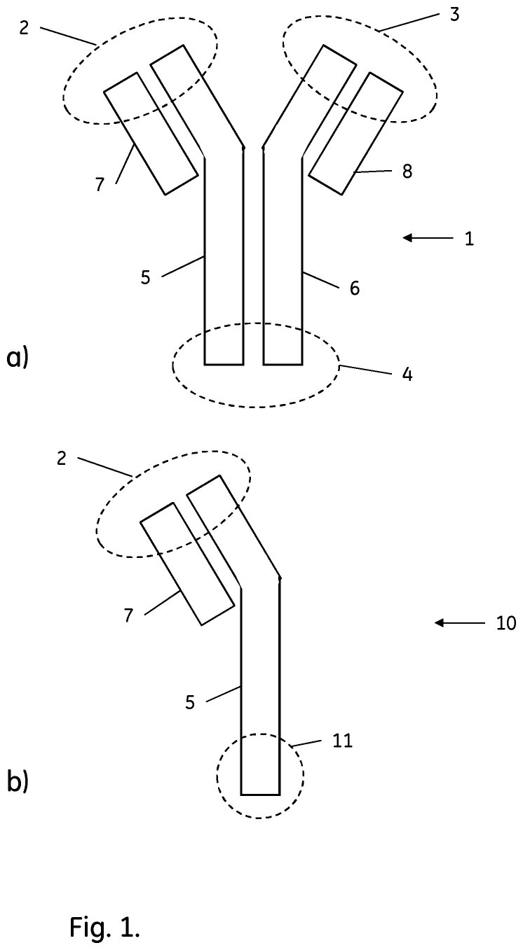

FIG. 1 shows schematically two Fc-comprising variants that can be separated by the methods of the invention. FIG. 1 a) shows a bispecific (heterodimeric) IgG antibody 1 with two different antigen-binding sites 2,3 and an Fc-region 4. The antibody comprises two heavy chains 5,6 and two light chains 7,8. In the expression process the cells generate half-antibodies 10, as in FIG. 1 b), comprising one heavy chain 5 and one light chain 7, forming one antigen-binding site 2 and one half 11 of the whole antibody Fc region. The Fc-region 4 binds strongly to Protein A and other Fc-binding affinity ligands. Even the half Fc region 11 binds to to Protein A and other Fc-binding affinity ligands, but less strongly, as it only has half as many interaction points. In addition to half-antibodies, the desired heterodimeric bispecific antibody will also be contaminated with homodimeric antibodies where both antigen-binding sites are identical. Many different bi- and multispecific antibodies have been constructed, as described in e.g. U Weidle et al in Cancer Genom. Proteom. 10, 1-18, 2013, and the cell cultures comprising them will contain different non-desired variants analogue to the description above, which can be separated from the desired variant by the methods of the invention.

### Examples

### Feed

The feed used in the examples was a cell culture supernatant containing a bispecific IgG antibody (heterodimer and homodimer mixture), the corresponding half-antibodies and other substances originating from the cell culture (host cell proteins, DNA etc.). The antibody concentration was 0.35 mg/ml, the pH 7.4 and the conductivity 12-14 mS/cm.

SEC Analysis

SEC analysis of the feed and the eluate fractions was done on an ÄKTAexplorer10 chromatography system (GE Healthcare) in an automatic fashion according to the following method:
Sample: 20 μl
Column: Superdex™ 200 Increase 30/300 (GE Healthcare), Vt=24 mL
Buffer: PBS
Flow: 1 ml/min

### Example 1. Screening with Elution Gradient pH 6.0-3.0

Three different Protein A resins were investigated; MabSelect SuRe LX, MabSelect SuRe pcc and MabSelect PrismA (all GE Healthcare). The feed was applied and eluted using a pH-gradient according to the protocol below. Fractions were collected and analyzed using SEC.
Sample: 3 ml Feed
Column: Tricorn™ 5/50 (GE Healthcare), bed height=4.8 cm, bed volume=1 ml
Buffer A1: PBS
Buffer A3: 25 mM Na-citrate pH=6.0
Buffer B1: 25 mM Na-citrate pH=3.0
Wash: 100% Buffer A3 10CV (column volumes)
Elution: 0-100% B1 in 20CV
Flow: 0.25 ml/min

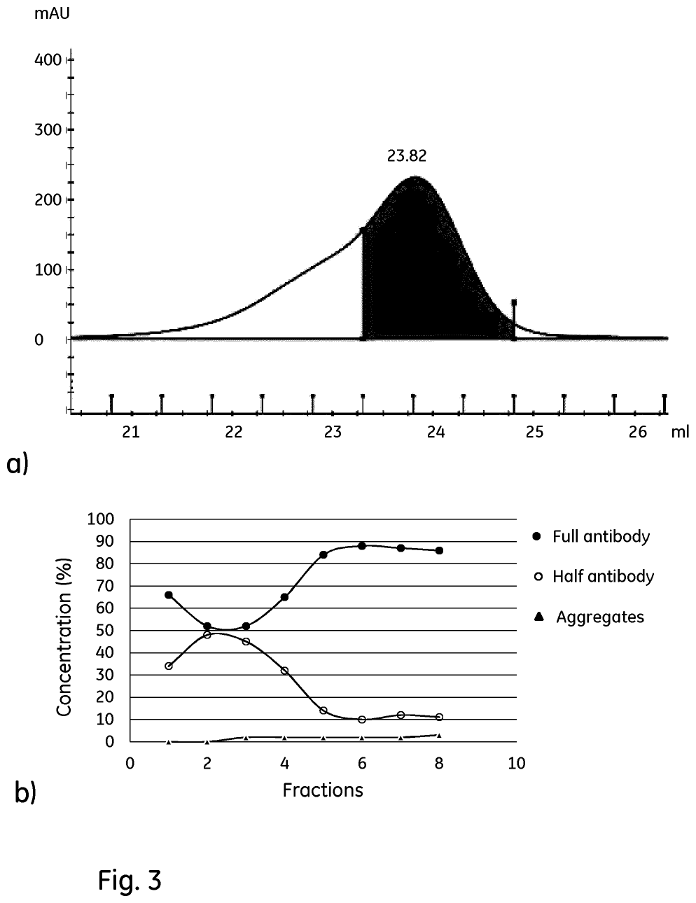

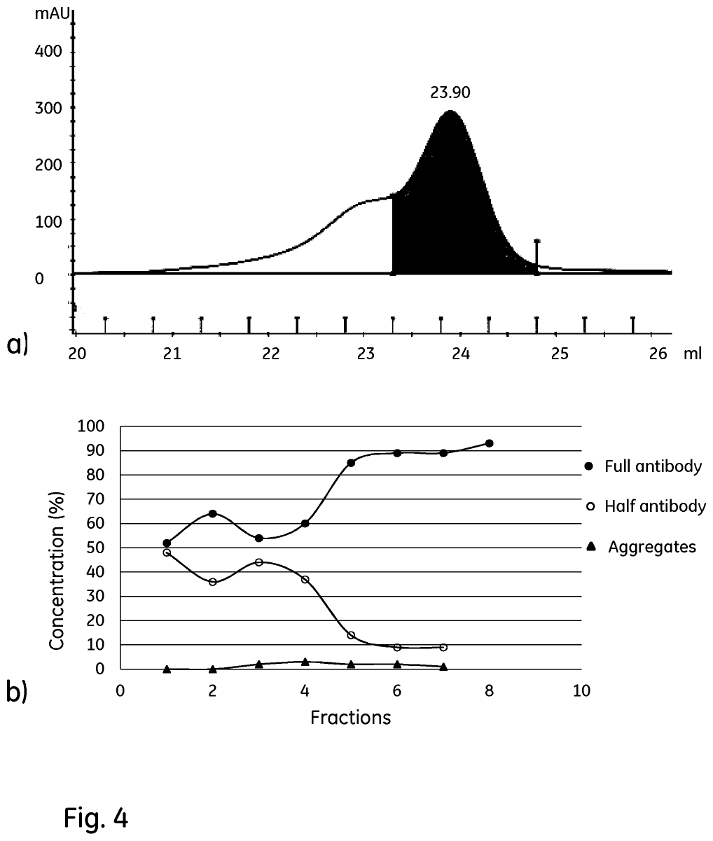

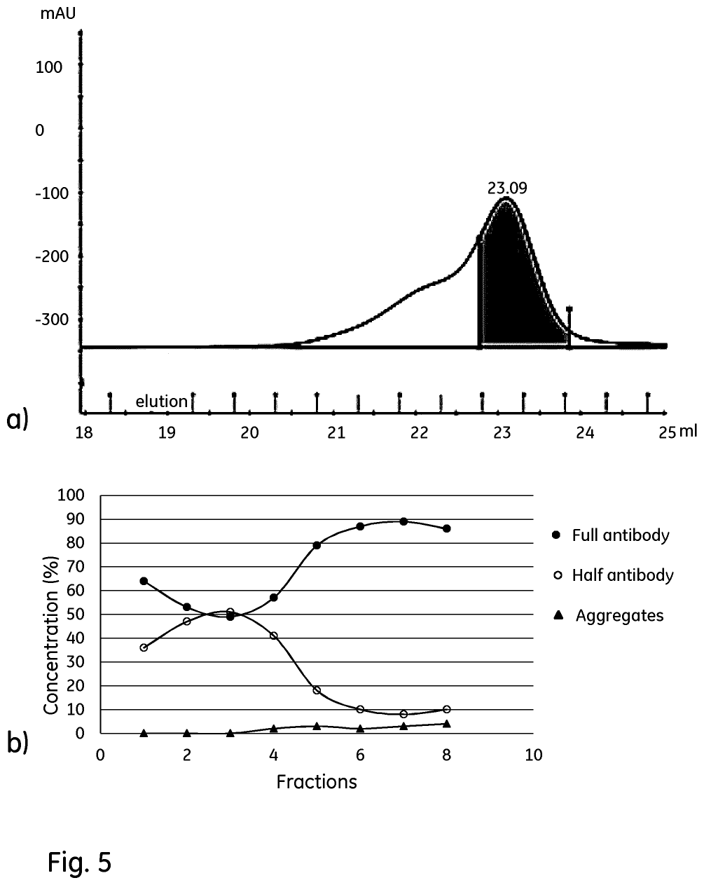

As shown in FIGS. 3-5, the three different resin products gave similar results, although MabSelect SuRe pcc (FIG. 4) and MabSelect PrismA (FIG. 5) showed a somewhat better resolution compared to MabSelect SuRe LX (FIG. 3). This may be related to their different particle sizes; MabSelect SuRe pcc (d50,v=50 μm) is the smallest closely followed by MabSelect PrismA (d50,v=60 μm) and then MabSelect SuRe LX (d50,v=85 μm).

### Example 2. Elution Gradient pH 5.0-3.0

The feed was applied to a MabSelect PrismA column and eluted using a pH-gradient according to the protocol below. Fractions were collected and analyzed using SEC.
Sample: 3 ml Feed
Column: Tricorn™ 5/50 (GE Healthcare), bed height=4.8 cm, bed volume=1 ml
Buffer A1: PBS
Buffer A3: 25 mM Na-citrate pH=6.0
Buffer B1: 25 mM Na-citrate pH=3.0
Wash: 100% Buffer A3 10CV
Elution: 30-100% B1 in 20CV
Flow: 0.25 ml/min

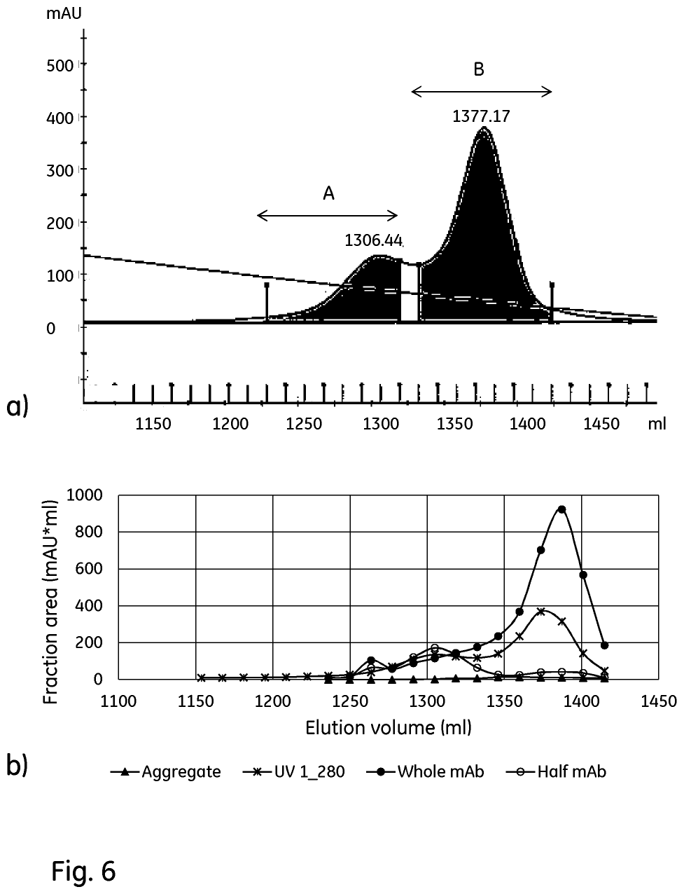

The results are shown in FIG. 6. Two pools were collected, A and B, where B contained the full mAb see FIG. 6 a) and b). The SEC analysis confirmed what we could see in the screening experiment, namely that a good resolution could be achieved between whole and half mAb. Judged from the SEC analysis it seemed as if the B-pool was well above 90% pure.

### Example 3. Elution Gradient pH 4.5-3.0

The feed was applied to a MabSelect PrismA column and eluted using a pH-gradient according to the protocol below. Fractions were collected and analyzed using SEC.
Sample: 3 ml Feed
Column: Tricorn™ 5/50 (GE Healthcare), bed height=4.8 cm, bed volume=1 ml
Buffer A1: PBS
Buffer A3: 25 mM Na-citrate pH=6.0
Buffer B1: 25 mM Na-citrate pH=3.0
Wash: 100% Buffer A3 10CV
Elution: 40-100% B1 in 20CV
Flow: 0.25 ml/min

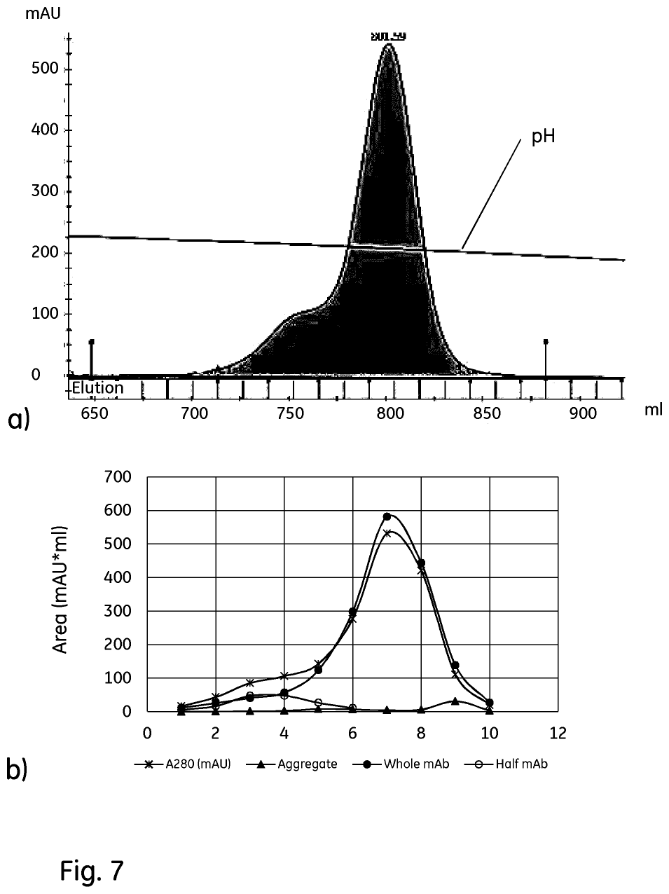

This experiment was essentially a repetition of Example 2, although with a somewhat shallower pH gradient. As shown in FIG. 7, the results were also very similar.

### Example 4. Subsequent Multimodal Cation Exchange Chromatography Step

The whole antibody fraction from the Example 2 eluate was loaded onto a Capto MMC ImpRes (GE Healthcare) multimodal cation exchange column and eluted with an increasing pH gradient.
Equilibration: 2 CV Buffer A, flow rate 1 mL/min
Sample appl: 1 mL (0.6 mg), flow rate 0.5 mL/min
Wash: 2 CV Buffer A, flow rate 0.5 mL/min.
Elution: 0-100% Buffer B in 20 CV
Wash: 2 CV Buffer B, flow rate 1 mL/min
CIP: 5 CV 1 M NaOH flow rate 0.66 ml/min (contact time 15 min)
Re-equilibration: 7 CV Buffer A
Sample: 0.6 mg of pool B (whole antibody fraction) from Example 2
Column: Tricorn 5/100, Capto MMC ImpRes
Wavelength: 280 nm
Buffer A: 10 mM Citrate+10 mM Phosphate+10 mM Tris pH 5
Buffer B: 10 mM Citrate+10 mM Phosphate+10 mM Tris pH 9

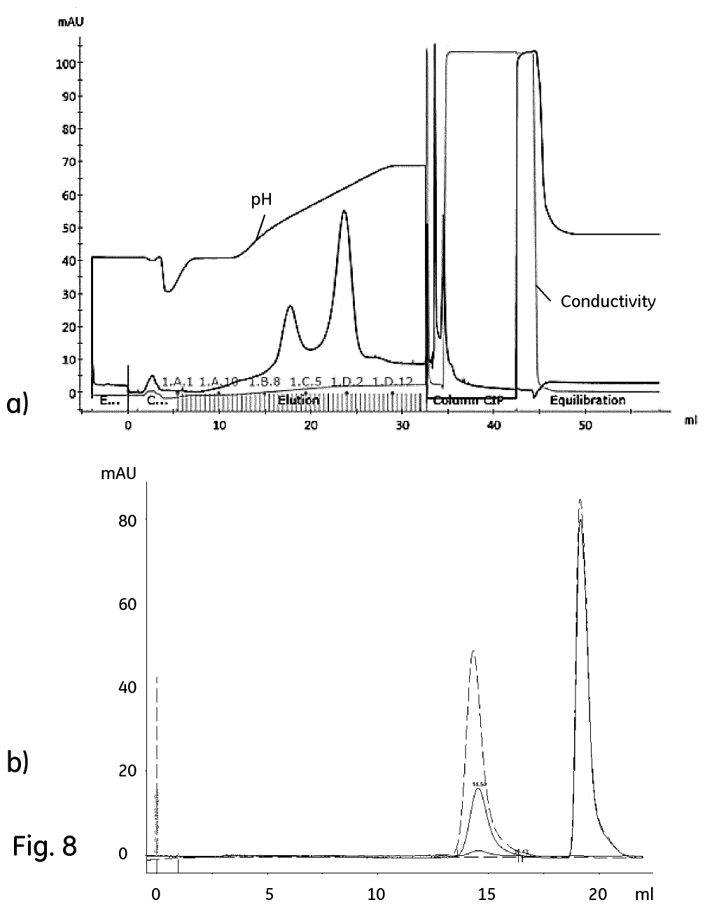

The results in FIG. 8 show that two well separated peaks were obtained. The SEC analysis showed that they had almost the same molecular weight (MW), the same as for a full mAb. This indicated that the two fractions should be two different species of whole mAb (presumably the heterodimer and homodimer) There was a small but repeatable difference in elution volume for the two species.

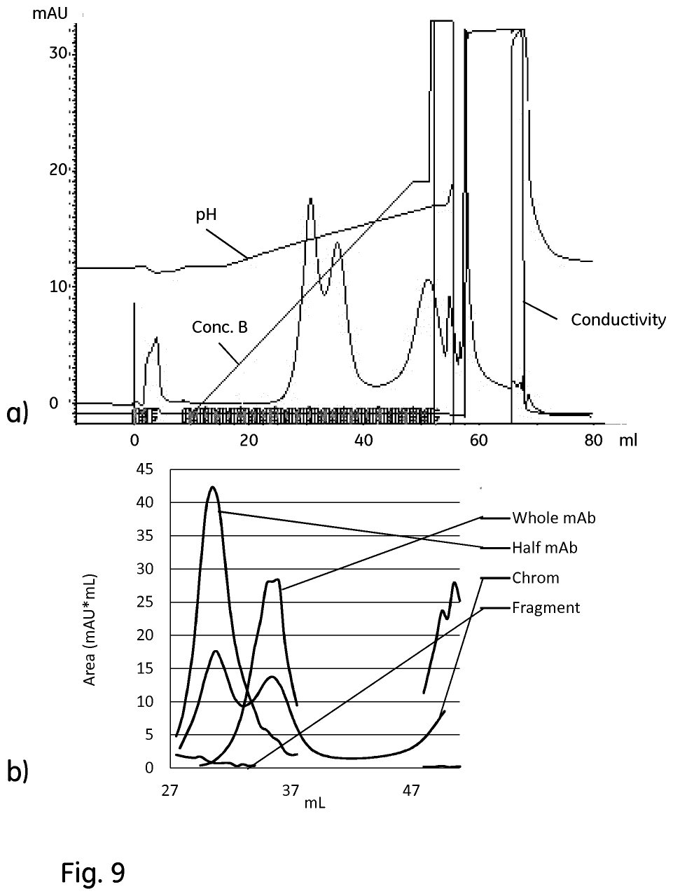

To be able to see if Capto MMC ImpRes could separate whole- from half mAb the experiment was repeated but with a mix of pool A and B from Example 2 (FIG. 9). Besides that, all variables were the same. When analysing the fractions with SEC it is clear that it is possible to separate half from whole mAb using Capto MMC ImpRes.

### Example 5. Subsequent Cation Exchange Chromatography Step

A mixture of the whole antibody and half-antibody fractions from the Example 2 eluate was loaded onto a Capto SP ImpRes (GE Healthcare) cation exchange column and eluted with an increasing pH gradient.
Equilibration: 5 CV Buffer A, flow rate 1 mL/min
Sample appl: 1.5 mL, flow rate 0.5 mL/min,
Wash: 3 CV Buffer A, flow rate 0.5 mL/min
Elution: 0-70% Buffer B in 30 CV
Wash: 2 CV Buffer B, flow rate 1 mL/min
CIP: 5 CV 1 M NaOH flow rate 0.7 ml/min (contact time 15 min)
Re-equlibration: 7 CV Buffer A
Frac. Vol: 0.5 ml
Sample: A mix of pool A and B from Example 2
Column: Tricorn 5/100, Capto SP ImpRes
Wavelength: 280 nm
Buffer A: 10 mM Citrate+10 mM Phosphate+10 mM Tris pH 5
Buffer B: 10 mM Citrate+10 mM Phosphate+10 mM Tris pH 9

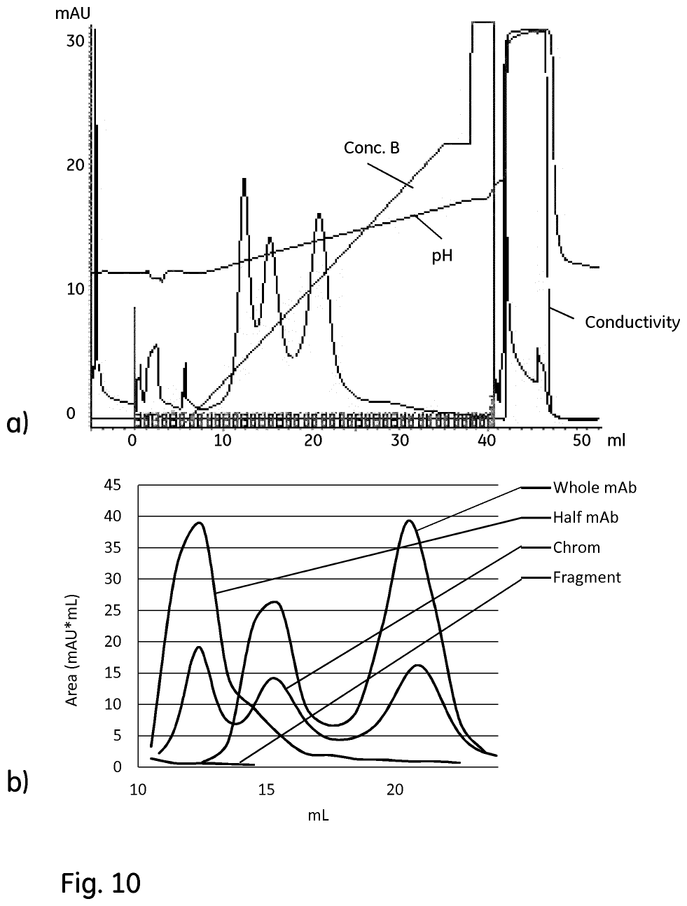

As shown in FIG. 10, whole- and half mAb could also be separated on Capto SP ImpRes with good recovery and resolution. The sample separated in three peaks that were analysed with SEC (FIG. 10b). The recovery was measured to >95%.

### Example 6. Subsequent Anion Exchange Chromatography Step

A mixture of the whole antibody and half-antibody fractions from the Example 2 eluate was titrated to pH 9 and loaded onto a Capto Q ImpRes (GE Healthcare) anion exchange column and eluted with a decreasing pH gradient.
Equilibration: 5 CV Buffer A, flow rate 1 mL/min
Sample appl: 5 mL (0.29 mg/mL), flow rate 0.5 mL/min,
Wash: 3 CV Buffer A, flow rate 1 mL/min
Elution: 0-100% Buffer B in 40 CV,
Wash: 2 CV Buffer B, flow rate 1 mL/min
CIP: 5 CV 1 M NaOH flow rate 0.7 ml/min (contact time 15 min)
Re-equlibration: 7 CV Buffer A
Wavelength: 280 nm
Sample: 4 ml pool A and 1 ml pool B from Example 2, titrated to pH=9 and filtrated 0.22 μm. Conc 0.29 mg/mL
Buffer A: 10 mM Citrate+10 mM Phosphate+10 mM Tris pH 9
Buffer B: 10 mM Citrate+10 mM Phosphate+10 mM Tris pH 5
Resin: HiTrap 1 ml Capto Q ImpRes

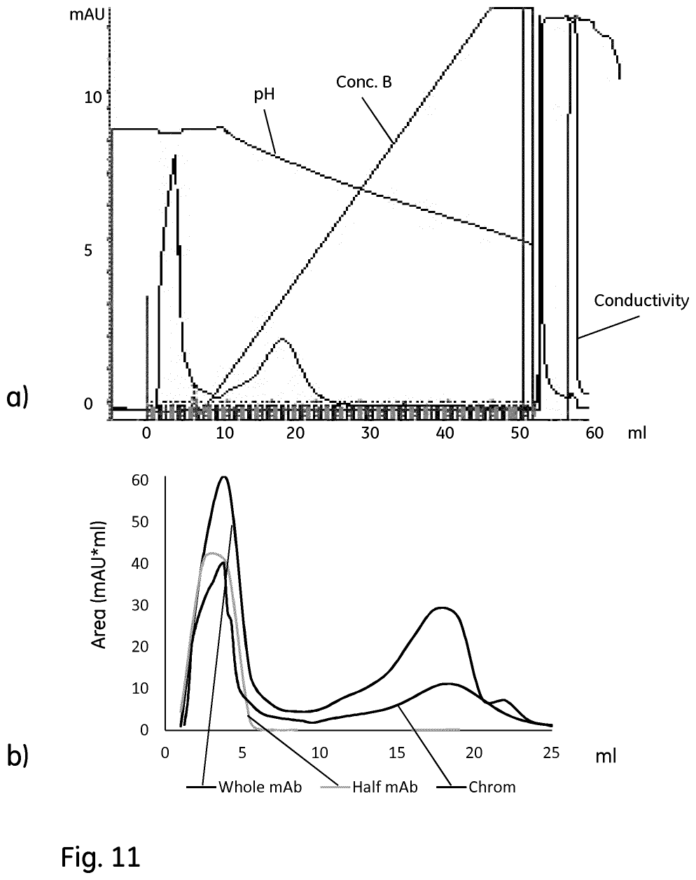

The results are shown in FIG. 11, indicating that one fraction was found in the flow-through. It turned out to be the half mAb and only one of the whole mAb variants.

### Example 7. Subsequent Hydrophobic Interaction Chromatography (HIC) Step

Two salt types, sodium sulphate and ammonium sulphate were evaluated at different concentrations. A solubility screening was performed in plate format to investigate the concentration where the mAb starts to precipitate. The salt concentration was lowered to be close to the point where the mAb did not bind, approximately 0.3 M sodium sulphate in phosphate buffer pH=7. The concentration of buffer salt was also reduced in order to lower the conductivity. Even using 3 mM sodium phosphate pH=7, the mAb did not elute. In the final protocol, MQ-water was used as B-solution since other investigated solutions did not manage to elute the bound mAb.

### Sample:

1.5 mL Pool 5.B1-5.B2 from Example 2, pH adjusted to 5, conc. 0.39 mg/mL and 1 mL Pool 5.B4-5.C5 from Example 2, conc. 0.66 mg/mL was mixed and buffer exchanged to 0.3 M Sodium Sulphate+21 mM Phosphate pH 7, using NAP-25 (GE Healthcare, single-use column containing Sephadex™ G-25 gel filtration resin).

The NAP-25 column was equilibrated with the buffer, 2.5 mL sample was added and eluted with 3.5 mL buffer. Calculated conc. after buffer exchange 0.36 mg/mL

### Solutions

A1: Buffer A: 0.3 M Sodium sulphate+25 mM phosphate pH 7.0.
B1: Buffer B: MQ
B2: CIP 1: 30% IPA
A2: CIP 2: 1 M NaOH
   The running conditions were;
Equilibration: 5 CV Buffer A, flow rate 1 mL/min
Sample injection: 3 mL buffer exchanged (0.3 M Sodium sulphate+21 mM phosphate pH 7.0) PrismA eluate, 0.25 mL/min (0.5 mL fractions were collected)
Wash 1: 5 CV Buffer A, flow rate 1 mL/min (0.5 mL fractions were collected)
Elution: 0-100% Buffer B in 20 CV, 0.25 mL/min (0.5 mL fractions were collected)
Wash 2: 5 CV Buffer B, flow rate 1 mL/min
CIP 1: 5 CV 30% IPA, flow rate 0.7 mL/min
Wash 3: 5 CV Buffer B, flow rate 1 mL/min
CIP 2: 5 CV 1 M NaOH, flow rate 0.7 mL/min
Re-equilibration: 5 CV Buffer A, flow rate 1 mL/min
Resins: HiTrap Capto Butyl ImpRes (GE Healthcare)

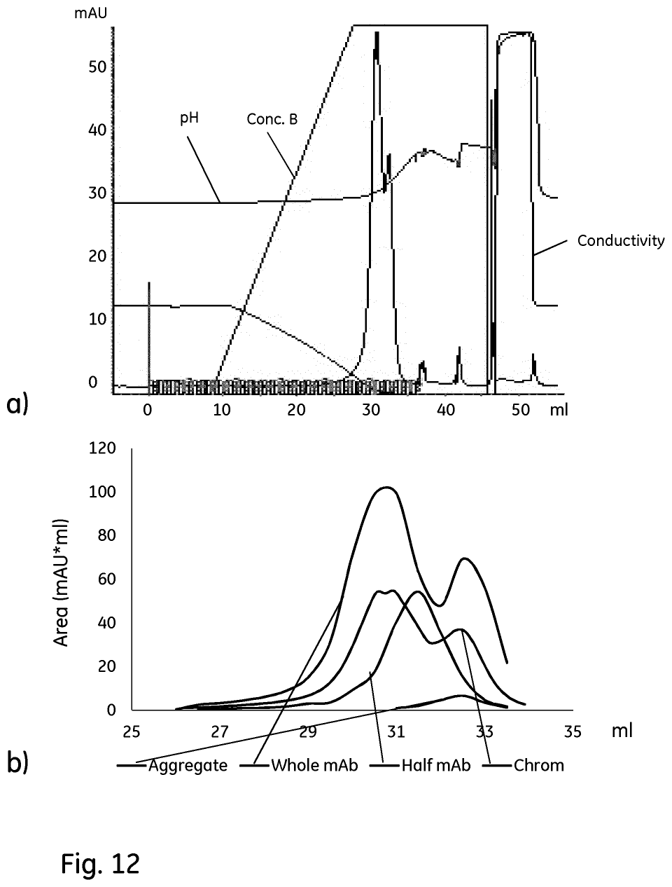

From the plot in FIG. 12 one can see that there is a small resolution of half- and whole mAb. There is also a resolution of the two variants of whole mAb.

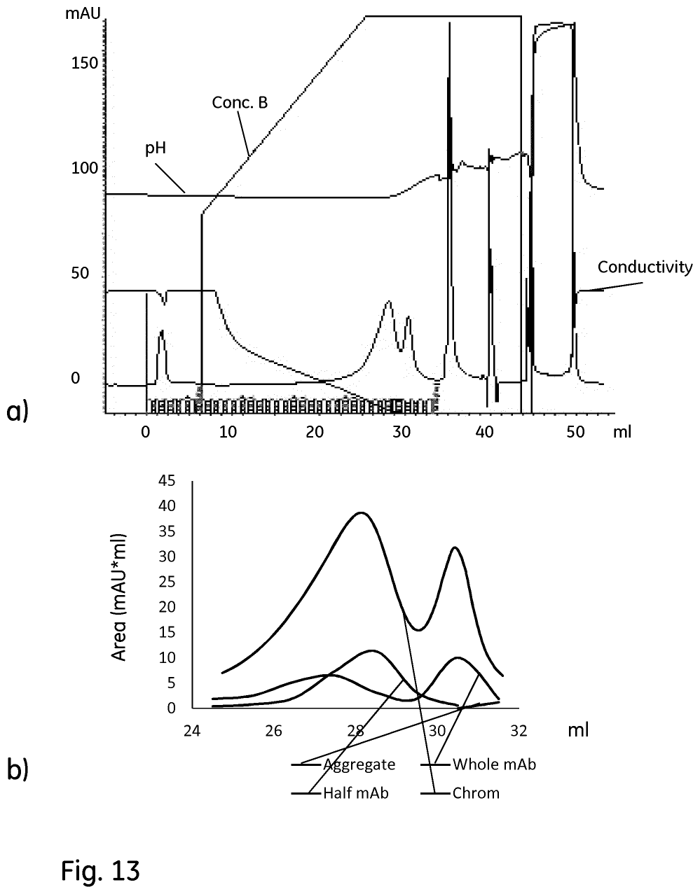

The same pattern could be seen using ammonium sulphate as salt (FIG. 13). With essentially the same conditions as above a resolution of half- and whole mAbs could be seen.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims. All patents and patent applications mentioned in the text are hereby incorporated by reference in their entireties, as if they were individually incorporated.

1. A method of separating immunoglobulin variants, comprising the steps of:
a) providing a column packed with an Fc-binding affinity chromatography resin;
b) loading a sample comprising at least two Fc-comprising immunoglobulin variants onto said column;
c) optionally washing said column with a washing liquid; and
d) conveying an eluent through said column to elute at least a target immunoglobulin variant from said column and recovering one or more eluate fractions comprising said target immunoglobulin variant in enriched form.

2. The method of claim 1, wherein in step d), said one or more eluate fractions comprise said target immunoglobulin variant in enriched form.

3. The method of claim 1, wherein in step d), the ratio of target Fc-comprising immunoglobulin variant concentration to total Fc-comprising immunoglobulin variant concentration is at least 50% higher than in said sample.

4. The method of claim 1, wherein one of said Fc-comprising immunoglobulin variants is an intact immunoglobulin, while another one of said Fc-comprising immunoglobulin variants is an Fc-comprising fragment of an immunoglobulin.

5. The method of claim 1, wherein one of said Fc-comprising immunoglobulin variants is an intact immunoglobulin with an Fc region and two Fab regions, while another one of said Fc-comprising immunoglobulin variants is a half-antibody with an Fc region and only one Fab region.

6. The method of claim 1, wherein said Fc-binding affinity chromatography resin is a Protein A resin, such as an alkali-stable Protein A resin.

7. The method of claim 1, wherein said Fc-binding affinity chromatography resin has a volume-weighted median particle diameter of less than 70 micrometers.

8. The method of claim 1, wherein said Fc-binding affinity chromatography resin has a volume-weighted median particle diameter of 40-65 micrometers.

9. The method of claim 1, wherein in step c) eluent with a gradient of decreasing pH is conveyed through said column.

10. The method of claim 9, wherein a start pH of said gradient is at least pH 4.5 and an end pH of said pH gradient is pH 3.5 or lower.

11. The method of claim 1, wherein in step b), the total amount of Fc-comprising immunoglobulin variants loaded onto said column exceeds the dynamic immunoglobulin binding capacity of said column.

12. The method of claim 1, wherein in step b), the total amount of Fc-comprising immunoglobulin variants loaded onto said column is at least 80 mg per ml column bed volume.

13. The method of claim 11, wherein said target Fc-comprising immunoglobulin variant is an intact immunoglobulin with an Fc region and two Fab regions and binds stronger to the column than Fc-comprising immunoglobulin fragments present in said sample, such as half-antibodies with an Fc region and a single Fab region.

14. The method of claim 1, wherein during step d) an elution fraction comprising a target Fc-comprising immunoglobulin variant is recovered, with a ratio of target Fc-comprising immunoglobulin variant concentration to total Fc-comprising immunoglobulin variant concentration at least 50% higher than in the loaded sample.

15. The method of claim 14, further comprising, after step d), a step e) of purifying said target Fc-comprising immunoglobulin variant in a subsequent chromatography step.

16. The method of claim 15, wherein said subsequent chromatography step is a cation exchange or multimodal cation exchange step.

17. The method of claim 16, where said cation exchange or multimodal cation exchange step is performed in bind-elute mode.

18. The method of claim 17, wherein elution in step e) is performed with a pH gradient, such as a continuous gradient or a step gradient.

19. The method of claim 17, wherein said subsequent chromatography step is a cation exchange step and elution in step e) is performed with a conductivity gradient, such as a continuous gradient or a step gradient.

20. The method of claim 16, wherein said subsequent chromatography step is an anion exchange or multimodal anion exchange step.

21. The method of claim 20, wherein elution in step e) is performed with a pH gradient, such as a continuous gradient or a step gradient.

22. The method of claim 16, wherein said subsequent chromatography step is a hydrophobic interaction chromatography (HIC) step.

23. The method of claim 22, wherein elution in step e) is performed with a decreasing salt gradient, such as a continuous gradient or a step gradient.

24. The method of claim 15, wherein step e) follows directly after step d).

25. The method of claim 15, wherein a step d′) of buffer adjustment is performed between step d) and step e).

26. The method of claim 15, wherein a step d″) of virus inactivation, virus filtration or ultrafiltration is performed between step d) and step e).
